(19) United States
(12) Patent Application Publication
(10) Pub. No.: US 2026/0185041 A1
(43) Pub. Date: Jul. 2, 2026

(54) EXTRACELLULAR MATRIX SUBSTITUTE IN A CELLULAR MICROCOMPARTMENT

(71) Applicant: TREEFROG THERAPEUTICS, PESSAC (FR)

(72) Inventors: Caroline Christine Marie ROYER, GRADIGNAN (FR); Joffrey Quentin MIANNE, PESSAC (FR); Maxime FEYEUX, TALENCE (FR)

(21) Appl. No.: 19/129,986

(22) PCT Filed: Nov. 15, 2023
(86) PCT No.: PCT/EP2023/081966

§ 371 Date: May 14, 2025

(30) Foreign Application Priority Data
Nov. 15, 2022 (FR) FR2211873

(51) Int. Cl. C12N 5/00; C07K 14/78

(52) U.S. Cl. CPC C12N 5/0062; C07K 14/78; C12N 2513/00; C12N 2533/74

(57) ABSTRACT

The invention relates to the field of three-dimensional cell culture and relates, in particular, to cellular microcompartments, without an extracellular matrix of animal origin, and/or derived from cancer cell lines, for the production of GMP-grade cells and tissues.

### TECHNICAL FIELD

The invention relates to the field of three-dimensional cell culture and relates, in particular, to cellular microcompartments, without an extracellular matrix of animal origin, and/or derived from cancer cell lines, for the production of GMP-grade cells and tissues.

### PRIOR ART

The discovery of induced pluripotent stem cells (iPS or iPSCs) by Prof. Yamanaka, has given new impetus to the field of cell culture. Clinical trials soon began, with the aim of treating diseases that were often rare and incurable with traditional medicine.

Historically, cells, including induced pluripotent stem cells, were cultivated in two dimensions (2D). Due to the limitations of the 2D cell culture, three-dimensional (3D) culture systems have been developed in recent years, making it possible to partially overcome the disadvantages of 2D culture.

Such systems are advantageously closer to in vivo natural systems, and can be used for numerous applications, notably cell therapy. The cells cultured in these systems may be of any type. It may involve both differentiated cells with different phenotypes, progenitor cells and stem cells.

A particularly suitable technology is that described in patent application WO 2018/096277 which describes three-dimensional microcompartments for stem cell culture, notably comprising cells, a Matrigel® like extracellular matrix layer and an outer layer of hydrogel.

Although it is a very promising technology, it still suffers from certain disadvantages in order to be able to be used in clinical conditions. To do this, both the cells and the three-dimensional culture systems from which the cells derive must comply with the regulations relating to Good Manufacturing Practices (GMP). However, most three-dimensional culture systems, such as cellular microcompartments, comprise an extracellular matrix of animal origin and/or derived from cancer cell lines, such as Matrigel®, also known under the name of Engelbreth-Holm-Swarm (EHS) matrix, which are incompatible with such regulations.

Thus, its extensive use in cell culture, notably that of iPS cells, is being called into question as Matrigel® is derived from mouse sarcomas, which are likely to introduce xenogenic contaminants into cells obtained from a culture comprising Matrigel®. Furthermore, the complexity of its mixing, the variability of biochemical and mechanical properties between batches and within batches mean that cell culture experiments lack reproducibility.

To consider the advent of cell therapies or the production of animal or vegetable cells for human or animal food consumption, based on this technology, there is a need to develop an alternative, a substitute making it possible to eliminate the presence of such an extracellular matrix, while retaining the possibility for the cells to adhere and grow satisfactorily.

In the course of their work, the inventors surprisingly discovered the use of a hydrogel having a high Young's modulus, intended to form the outer envelope or the outer layer of a cellular microcompartment combined with a second hydrogel having a Young's modulus less than that of the outer layer, intended to form a layer or a mesh in the inner part of the microcompartment, on which at least one cell can be housed and grow. Advantageously, the present invention makes it possible to obtain cells in large quantities as well as rapid growth and a low mutation rate. Such results thus make it possible to envisage its use in the production of three-dimensional cellular microcompartments for human and veterinary use.

Studies have certainly described the use of synthetic matrix, including in three dimensions. However, cellular microcompartments comprising two hydrogels each with a different Young's modulus are not described, in particular when the Young's modulus of the hydrogel of the outer layer is strictly greater than that of the hydrogel constituting the layer or the mesh of the inner part. However, such a structure is complex to implement, so that the outer layer is sufficiently stiff to form the outer envelope of the cellular microcompartment and thus protect the cell contents; whereas, the inner part of the cellular microcompartment, delimited by the outer envelope of said microcompartment, comprising at least said hydrogel layer or mesh, has particular rheological properties, as well as a mechanical strength allowing it to provide the cells with an environment conducive to their dispersal, proliferation and migration.

Also, to meet this need for a cellular microcompartment without a non-GMP extracellular matrix such as Matrigel®, having rheological properties suitable for cell protection and growth within the microcompartment, the invention proposes a three-dimensional culture system based on a three-dimensional cellular microcompartment comprising at least two hydrogels having distinct Young's modulus, in particular the Young's modulus of the hydrogel of the outer layer is strictly greater than that of the hydrogel comprised in the inner part and composing the layer or the mesh in the microcompartment.

### SUMMARY OF THE INVENTION

Thus, the invention relates to a new three-dimensional cellular microcompartment comprising:
an outer layer of hydrogel, and
an inner part comprising at least one cell and at least one layer or a mesh of hydrogel of plant or synthetic origin,
   wherein the Young's modulus of the hydrogel in the inner part is strictly less than the Young's modulus of the hydrogel in the outer layer.

The invention thus relates to the use of two specific hydrogels, one intended to form the outer layer and the second intended to form the layer or the mesh in the inner part, it being understood that said inner part is delimited by the outer layer of hydrogel and the Young's modulus of the hydrogel of the inner part is strictly less than the Young's modulus of the hydrogel of the outer layer.

On the one hand, the outer layer of hydrogel forms the protective outer envelope, and therefore constitutes the capsule, and on the other hand, the less stiff, looser layer or mesh of the inner part allows cell growth, given the particularly suitable rheological properties of the hydrogel of said layer or mesh of the inner part.

The microcompartment according to the invention is thus composed of two distinct hydrogels. The hydrogel constituting the outer layer having a Young's modulus strictly greater than the Young's modulus of the hydrogel constituting the layer or the mesh of the inner part, which improves the protection of the cells and cellular aggregates present in the cellular microcompartment.

Indeed, advantageously, the outer layer provides a first protective envelope. This effect is enhanced by the at least one hydrogel layer or mesh in the inner part, which provides an additional protective mesh for the cells and aggregates present in the microcompartment while being sufficiently loose to allow cell migration and amplification and maintain their pluripotent character. Conversely, the known prior art, composed of a single layer of hydrogel, is either too stiff, preventing cell migration and amplification and inducing cell apoptosis, or on the contrary, too loose and therefore incompatible with culture in a bioreactor. Indeed, the mechanical constraints of culture in a bioreactor, notably due to the high shear forces generated during culture require a suitable cellular microcompartment.

The outer layer of hydrogel having a high Young's modulus therefore forms the outer envelope of the microcompartment or of the capsule according to the invention, allowing the contents of the capsule to be protected from the external environment, notably when the capsules are cultivated in a bioreactor. This is further enhanced by the presence of the hydrogel layer or mesh on the inner part of said microcompartment. Advantageously, the hydrogel layer or mesh of the inner part is at least partially juxtaposed to the inner face of the outer layer.

According to a preferred object of the invention, the Young's modulus of the hydrogel of the mesh or of the layer in the inner part is between 0.01 and 200 kPa, more preferentially between 0.1 and 60 kPa, even more preferentially between 0.1 and 5 kPa.

Preferentially, the Young's modulus of the hydrogel of the outer layer is greater than 10 kPa, more preferentially greater than 60 kPa, even more preferentially greater than 100 kPa.

By way of example, when the Young's modulus of the hydrogel present in the inner part is 10 kPa, preferentially 60 kPa, more preferentially 100 kPa, the Young's modulus of the hydrogel in the outer layer is necessarily strictly greater than 10 kPa, preferentially greater than 60 kPa, more preferentially greater than 100 kPa.

According to a particularly preferred embodiment of the invention, the hydrogel of the outer layer and/or of the inner part constituting the microcompartment is composed or exclusively consists of alginate.

Alginates are monomers of β-D-mannuronic acid (M units) and α-L-guluronic acid (G units) with (1-4) bonds, which vary in amount and in sequential distribution along the polymer chain. Divalent cations such as Ca2+ bind cooperatively between the G blocks of adjacent alginate chains, creating inter-chain ionic bridges that cause the gelification of aqueous alginate solutions.

When the hydrogel included in the inner part is alginate, the concentration of the alginate solution intended to form the layer or the mesh of alginate in the inner part of the microcompartment, is preferentially between 0.25 and 2%, more preferentially between 0.25 and 1%, even more preferentially 0.5% (plus or minus 0.1%). When the concentration of the alginate solution intended to form the layer or the mesh of alginate in the inner part of the microcompartment is 0.5%, the viscosity of the alginate is preferentially 3 mPa/s.

Advantageously, the inner part also comprises at least one culture medium.

According to a preferred object of the invention, the hydrogel layer or mesh of the inner part is arranged between the outer layer of hydrogel and said at least one cell layer. More preferentially, the inner part comprises at least one layer of cells.

According to another preferred object, the molecular weight of the hydrogel of the inner part is strictly less than the molecular weight of the hydrogel of the outer layer. More preferentially, the alginate of the inner part has a molecular weight less than or equal to 75 kDa and/or the alginate of the outer layer has a molecular weight of between 150 and 250 kDa, which notably makes it possible to have an envelope that is sufficiently stiff to protect the contents of the capsule, and a looser layer or a mesh of the inner part, capable of acting as an extracellular matrix substitute for the cells present in the capsule and reinforcing the protective effect. Lower molecular weight alginate having shorter chains enables lower polymerization and viscosity of the gel, and as a result, a looser hydrogel allowing cells to migrate, grow and form a cellular aggregate, or even one or more cysts.

According to another object, the hydrogel layer or mesh of the inner part may comprise other constituents. Thus, said layer or mesh of hydrogel preferentially comprises at least one peptide sequence, more preferentially a peptide sequence of interest capable of interacting with the cells constituting notably the layer of cells present in the microcompartment according to the invention, this making it possible to improve the adhesion and the survival of the cells within the microcompartment. By way of example, the peptide sequence can be a peptide or a protein, preferentially the peptide sequence is derived from an extracellular matrix protein selected from collagen, fibronectin, laminin, etc. More preferentially, the peptide sequence is an RGD motif or a YIGSR motif.

The RGD motif enables cell adhesion by virtue of the interaction of the RGD peptide sequence with the integrins on cells. An RGD motif means a peptide with an RGD sequence that may comprise one or two other residues before and after the RGD motif to enhance cell detection of the RGD motif.

The YIGSR motif enables cell adhesion by virtue of the interaction of the YIGSR peptide sequence with the laminin receptors on cells. An YIGSR motif means a peptide with an YIGSR sequence that may comprise one or two other residues before and after the YIGSR motif to enhance cell detection of the YIGSR motif.

Advantageously, the layer or the mesh of hydrogen of the inner part also comprises at least a second hydrogel distinct from the first hydrogel of the inner part, more preferentially it is selected from fibrin, laminin, collagen, fibronectin, entactin and hyaluronic acid.

According to another object of the invention, the micro-compartment is preferentially closed. The microcompartment according to the invention may also be in different shapes, preferentially in the shape of an ovoid, a cylinder, a spheroid, a sphere or a teardrop.

Also, the microcompartment according to the invention is composed of three major constituents, an outer layer of hydrogel forming the envelope of said microcompartment with a high Young's modulus, a hydrogel layer or mesh with a low Young's modulus in the inner part of the cellular microcompartment, said layer or mesh having a Young's modulus strictly less than that of the hydrogel intended to form the outer layer, said layer or mesh being intended to serve as a substitute for the extracellular matrix, notably to replace Matrigel®. Finally, said inner part comprises at least one cell.

The cells present in the inner part advantageously constitute a layer of cells, the cells can then be of all cell types, preferentially said cells are not cells derived from human embryo or requiring the destruction of a human embryo, more preferentially, the cells are selected from human, animal and plant eukaryotic cells, even more preferentially pluripotent cells, progenitors, differentiating cells and differentiated cells.

When the microcompartment comprises the three constituents previously described, said microcompartment preferentially comprises at least one layer of cells and at least one lumen. When the microcompartment comprises at least one lumen, the cell layer, the hydrogel layer or mesh of the inner part and the outer layer are preferentially organized successively around said lumen.

Preferentially, the inner part of the microcompartment comprises at least one three-dimensional cell aggregate and/or a cellular microtissue.

According to another aspect, the invention also relates to a microcompartment assembly, wherein said assembly comprises at least one microcompartment according to any one of the preceding embodiments.

The microcompartment according to the invention or the microcompartment assembly according to the invention, is also particularly suitable for use in cell culture, in particular in three-dimensional cell culture, allowing the production of cells of interest in large quantities, microtissues, or even organoids of interest, suitable for use, for example, in cell therapy. Also, the invention also relates to a microcompartment according to the invention or a microcompartment assembly according to the invention, for use as a medicament.

According to one variant, the invention also relates to a use of the microcompartment according to the invention or of a microcompartment assembly according to the invention to manufacture microtissues. It is understood that in this particular embodiment, the microtissues are not implanted in a human being or an animal. By way of example, they can be used as an ex-vivo model.

On the other hand, the microcompartment according to the invention can be produced in different ways, however according to a particular aspect, the invention relates to a method for preparing the cellular microcompartment according to the invention, comprising the following steps:
a. mixing cells, optionally previously incubated in a culture medium,
b. encapsulating the mixture from step (a) in a hydrogel intended to form the outer layer;
c. cultivating the capsules obtained in step (b) in a culture medium, preferentially in a bioreactor, preferentially for at least 1 day, even more preferentially from 3 to 50 days, and
d. optionally recovering the cellular microcompartments obtained,
characterized in that, in step (a) and/or in step (b), a hydrogel solution of plant or synthetic origin is added, whose Young's modulus is strictly less than the Young's modulus of the hydrogel used to form the outer hydrogel layer.

According to a particular object of the invention, the step (b) comprises the following sub-steps:
bringing the mixture from step (a) into contact with a hydrogel solution intended to form the outer layer (i) to form at least one drop, and
collecting the drops in a calcium bath capable of stiffening said hydrogel solution to form the outer layer of each microcompartment.

In a particularly preferred way, step b) is carried out by simultaneous co-injection of the hydrogel solution intended to form the outer layer (i), of the mixture from step a) optionally comprising the hydrogel solution of plant or synthetic origin whose Young's modulus is strictly less than that of the hydrogel used to form the outer layer (ii), and optionally of an intermediate solution (iii) optionally comprising the hydrogel solution of plant or synthetic origin whose Young's modulus is strictly less than that of the hydrogel used to form the outer layer; said co-injection is carried out concentrically via a microfluidic or millifluidic injector forming a jet at the injector outlet made up of the mixture of said solutions, said jet breaking up into drops.

Advantageously, the final opening diameter of the microfluidic injector is between 50 and 800 μm, preferentially between 80 and 240 μm, and the flow rate of each of the solutions is between 0.1 and 2000 mL/h, preferentially between 10 and 2000 mL/h, more preferentially between 10 and 150 mL, even more preferentially between 11 and 100 mL/h.

According to a variant of the method according to the invention, step b) is carried out by simultaneous co-injection of the hydrogel solution intended to form the outer layer (i), of the mixture from step a) optionally comprising the hydrogel solution of plant or synthetic origin whose Young's modulus is strictly less than that of the hydrogel used to form the outer layer (ii), and optionally of an intermediate solution (iii) optionally comprising the hydrogel solution of plant or synthetic origin whose Young's modulus is strictly less than that of the hydrogel used to form the outer layer; said co-injection is performed concentrically via a microfluidic or millifluidic injector, said injector comprising a tip, said tip being in contact with a calcium solution, forming a jet at the injector outlet consisting of the mixture of said solutions, said jet forming a tube.

When said injector comprises a tip, said tip being in contact with the calcium solution, the final opening diameter of the microfluidic injector is preferentially between 50 and 1000 μm, more preferentially between 80 and 300 μm, and the flow rate of each of the solutions is between 1 and 100 mL/h.

According to a final aspect, the invention relates to a kit, said kit comprising a hydrogel solution intended to form the outer layer of the microcompartment (i) and a hydrogel solution of plant or synthetic origin whose Young's modulus is strictly less than that of the hydrogel used to form the outer layer (ii), preferentially an alginate, optionally an intermediate solution (iii), preferentially a sorbitol solution.

Finally, the invention also relates to the use of the kit intended for preparing a microcompartment according to the invention.

Other features and advantages will become apparent from the detailed description of the invention, the examples and the figures that follow.

### BRIEF DESCRIPTION OF THE FIGURES

FIG. 1 shows a cellular microcompartment 1 according to a particular embodiment of the invention comprising an outer layer of hydrogel 2, and an inner part 3 delimited by said outer layer 2. Said inner part 3 comprising a hydrogel layer or mesh 30 whose Young's modulus is strictly less than that of the hydrogel of the outer layer 2, a cell layer 31, and a lumen 32. Said layer or mesh 30 is arranged between the inner face 20 of the outer layer of hydrogel 2 and the cell layer 31.

FIG. 2 shows one embodiment of the invention, in which the 0.5% alginate solution is mixed with sorbitol and introduced into a first injection line “IS”. The other two solutions, comprising either 2% alginate “A” to form the outer layer, or cells suspended in culture medium “CS”, are present in the other two injection lines. The three lines are then co-injected, using a micro-fluidic injector, with the various solutions forming a jet, breaking up into drops in the CaCl2 bath, said bath stiffening the outer layer from the 2% alginate solution and thus forming the capsule.

FIG. 3 shows a second embodiment of the invention on panel A, in which the 0.5% alginate solution is mixed with sorbitol and introduced into a first injection line “IS”. The other two solutions comprising either 2% alginate “A” or cells suspended in culture medium “CS”, are present in the other two injection lines. The three lines are then co-injected, using a micro-fluidic injector, the tip of which is brought into contact with the CaCl2 bath, forming a jet, taking the form of a tube in the CaCl2 bath, said bath stiffening the outer layer from the 2% alginate solution and thus forming the tube. Panel B is a 4× magnification image taken using an EVOS microscope of cells in tubes comprising 0.5% alginate on D4 and D5.

FIG. 4 shows the percentage of empty capsules, that is without cells or cysts, in capsules without an exogenous extracellular matrix (outside the scope of the invention), Matrigel®-based capsules (outside the scope of the invention), and 0.5% alginate-based capsules as a matrix substitute (invention)

FIG. 5a is a 4× magnification image taken using a transmitted-light microscope of cells in capsules without an exogenous extracellular matrix (outside the scope of the invention) on DO.

FIG. 5b is a 4× magnification image taken using a transmitted-light microscope of cells in Matrigel®-based capsules (outside the scope of the invention) on DO.

FIG. 5c is a 4× magnification image taken using a transmitted-light microscope of cells in 0.5% alginate-based capsules (invention) on DO.

FIG. 6a is a 4× magnification image taken using a transmitted-light microscope of cells in capsules without an exogenous extracellular matrix (outside the scope of the invention) on D5.

FIG. 6b is a 4× magnification image taken using a transmitted-light microscope of cells in Matrigel®-based capsules (outside the scope of the invention) on D5.

FIG. 6c is a 4× magnification image taken using a transmitted-light microscope of cells in 0.5% alginate-based capsules (invention) on D5.

FIG. 7 shows the amplification and the pluripotency on D5 of cells present in capsules without an exogenous extracellular matrix (outside the scope of the invention), Matrigel®-based capsules (outside the scope of the invention), and 0.5% alginate-based capsules (invention).

FIG. 8 shows a three-dimensional cellular microcompartment 1 according to one embodiment of the invention, comprising an outer layer of hydrogel 2 forming a hollow cavity, that is the inner part 3 of the microcompartment according to invention 1. The hollow cavity 3 comprises a hydrogel mesh 4 in contact with the outer layer 2 and a plurality of cells organized in 3D forming a cyst 5, said cyst 5 comprising a lumen 6 in the center thereof.

FIG. 9 shows the amplification on D5 of cells present in 0.5% alginate-based capsules, 0.5% alginate-based capsules functionalized with at least one YIGSR motif, said motif further comprising a spacer consisting of 6, 9, 12 glycine residues.

FIG. 10 shows the pluripotency on D5 of cells present in 0.5% alginate-based capsules, 0.5% alginate-based capsules functionalized with at least one YIGSR motif, said motif further comprising a spacer consisting of 6, 9, 12 glycine residues.

FIG. 11 shows the amplification results on D5 of differentiated cells, namely cells of the endoderm (panel A) and mesoderm (panel B) present in 0.5% alginate capsules.

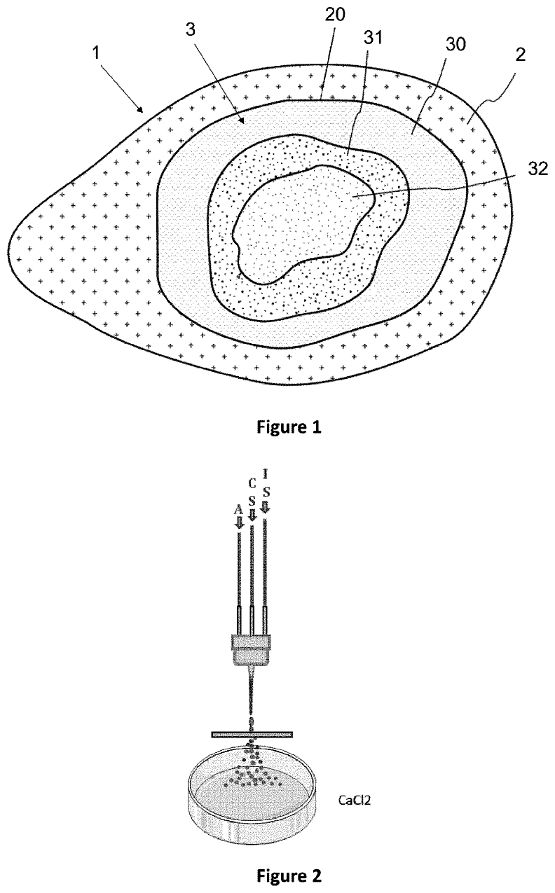

### DETAILED DESCRIPTION OF THE INVENTION

### Definition

“Hydrogel of plant or synthetic origin” means a hydrogel that is not of animal origin and/or derived from cancer cell lines, such as Matrigel®.

For the purposes of the invention, “hydrogel having a high Young's modulus” means a hydrogel having a Young's modulus strictly greater than the Young's modulus of the hydrogel constituting the layer or the mesh in the inner part of the cellular microcompartment according to the invention. Also, the Young's modulus of the hydrogel of the outer layer is strictly greater than the Young's modulus of the hydrogel present in the inner part constituting the layer or the mesh intended to replace an extracellular matrix of animal origin such as Matrigel®.

Conversely, “low Young's modulus” means a hydrogel having a Young's modulus strictly less than the Young's modulus of the hydrogel intended to form the outer layer of the microcompartment according to the invention.

For the purposes of the invention, “high-molecular-weight hydrogel” means a hydrogel of higher molecular weight constituting the outer layer of the cellular microcompartment according to the invention with respect to the hydrogel constituting the layer or the mesh of the inner part of the cellular microcompartment which is of lower molecular weight. Also, the molecular weight of the hydrogel of the outer layer is greater than that present in the inner layer.

Conversely, for the purposes of the invention, “low-molecular-weight hydrogel” means a hydrogel of lower molecular weight, that is of lower molecular weight than the hydrogel constituting the outer layer of the cellular microcompartment, said low-molecular-weight hydrogel constituting the layer or the mesh of the inner part of the cellular microcompartment according to the invention. Also, the molecular weight of the hydrogel present in the inner part is less than that of the outer layer.

For the purposes of the invention, “microcompartment” or “capsule” also means a partially or entirely closed three-dimensional structure, containing a plurality of cells. This is formed from a matrix of polymer chains, for example alginate, swollen by a liquid and preferentially water. The structure thus consists of a stiffened outer layer of hydrogel forming a hollow cavity or inner part comprising at least one cell, preferentially a plurality of cells and a layer or mesh of hydrogel suitable for cell culture and the growth of said cells.

For the purposes of the invention, “drop” also means a three-dimensional structure formed from at least one liquid solution comprising the constituents of a non-stiffened hydrogel (polymerization precursors, non-crosslinked or partially crosslinked polymer chains, etc.), of hydrogel precursor elements. Also, the drop constitutes a transient state between the co-injection of the various components and the microcompartment according to the invention.

For the purposes of the invention, “differentiated” cells means cells which have a particular phenotype, as opposed to pluripotent stem cells which are not differentiated or progenitor cells which are undergoing differentiation.

For the purposes of the invention, “human cells” means human cells or immunologically humanized non-human mammalian cells. Even when this is not specified, the cells, stem cells, progenitor cells and tissues according to the invention consist of or are obtained from human cells or from immunologically humanized non-human mammalian cells.

For the purposes of the invention, the term “mutant cell” refers to a cell carrying at least one mutation.

For the purposes of the invention, “progenitor cell” means a stem cell that is already engaged in cell differentiation but that has not yet differentiated.

For the purposes of the invention, “embryonic stem cell” means a pluripotent stem cell of cells derived from the internal cell mass of the blastocyst. The pluripotency of embryonic stem cells can be evaluated by the presence of markers such as the transcription factors OCT4, NANOG and SOX2 and surface markers such as SSEA4/5, Tra-1-60 and Tra-1-81. The embryonic stem cells used in the context of the invention are obtained without destroying the embryo from which they originate, for example using the technique described in Chang et al. (Cell Stem Cell, 2008, 2(2): 113-117). Optionally, embryonic stem cells from humans can be excluded.

For the purposes of the invention, “pluripotent stem cell” or “pluripotent cell” means a cell which has the capacity to form all the tissues present in the entire organism of origin, without however being able to form an entire organism per se. Human pluripotent stem cells can be called hPSC in the context of the present invention. These may in particular be induced pluripotent stem cells (iPSC or hiPSC for human induced pluripotent stem cells), embryonic stem cells or MUSE cells (for “multilineage-differentiating stress enduring”).

For the purposes of the invention, “induced pluripotent stem cell” means a pluripotent stem cell induced to become pluripotent by genetic reprogramming of differentiated somatic cells. These cells are notably positive for pluripotency markers, such as staining with alkaline phosphatase and expression of the proteins NANOG, SOX2, OCT4 and SSEA4/5. Examples of methods for obtaining induced pluripotent stem cells are described in the articles by Yu et al. (Science 2007, 318 (5858): 1917-1920), Takahashi et al (Cell, 207, 131(5): 861-872) and Nakagawa et al. (Nat Biotechnol, 2008, 26(1): 101-106).

For the purposes of the invention, “layer of cells” or “cellular base layer” is understood to mean a plurality of cells forming a layer or a base that can be structured around a lumen, it may for example be a cellular tissue or a microtissue or a three-dimensional grouped culture. The thickness of the layer of cells can be variable. This layer of cells is organized in three dimensions in the microcompartment.

For the purposes of the invention, “tissue” or “biological tissue” has the common meaning for tissue in biology, that is the intermediate organization level between the cell and the organ. A tissue is an assembly of similar cells and of the same origin (most commonly derived from a common cell line, although they can originate in the association of distinct cell lines), grouped into a cluster, network or bundle (fiber). A tissue forms a functional assembly, that is to say that its cells contribute to the same function. Biological tissues regenerate regularly and are assembled together to form organs.

For the purposes of the invention, “lumen” means a volume of aqueous solution topologically surrounded by cells. Preferentially, its content is not in diffusive equilibrium with the volume of convective liquid present outside the microcompartment.

### Microcompartment According to the Invention

The object of the present invention is a three-dimensional cellular microcompartment comprising:
an outer layer of hydrogel, and
an inner part comprising at least one cell and at least one layer or a mesh of hydrogel of plant or synthetic origin,
   wherein the Young's modulus of the hydrogel of the layer or of the mesh of the inner part is strictly less than the Young's modulus of the hydrogel of the outer layer.

In the context of the invention, the inner part is delimited by the outer layer of hydrogel, said hydrogel having a Young's modulus strictly greater than the Young's modulus of the hydrogel forming the layer or the mesh of the inner part, replacing the extracellular matrix of animal origin, such as Matrigel®. The present invention thus relates to a three-dimensional cellular microcompartment comprising two distinct hydrogels, one forming the outer layer forming a hollow cavity and the second the layer or the mesh present in the inner part of the microcompartment, that is the hollow cavity.

On the one hand, the outer layer of hydrogel forms the protective outer envelope, and therefore constitutes the capsule, and on the other hand, the less stiff, looser layer or mesh of the inner part allows cell growth, given its rheological properties particularly suitable for cell growth and survival. To achieve this, in the context of the invention, the Young's modulus of the hydrogel of the inner part is strictly less than the Young's modulus of the hydrogel of the outer layer.

Such a microcompartment according to the invention composed of two distinct hydrogels, wherein the hydrogel constituting the outer layer whose Young's modulus is strictly greater than the Young's modulus of the hydrogel constituting the layer or the mesh of the inner part improves the protection of cells and/or cell aggregates present in the microcompartment, in particular in the context of culture in a bioreactor, resulting in high shear forces.

Advantageously, the outer layer provides a first protective envelope, reinforced by the addition of at least one layer or a mesh of hydrogel, which provides an additional protective mesh for the cells and aggregates present in the microcompartment while being sufficiently loose to allow cell migration and amplification and maintaining their pluripotent character. Conversely, the known prior art, composed of a single layer of hydrogel, is either too stiff, preventing cell migration and amplification and inducing apoptosis, or on the contrary, not stiff enough and therefore incompatible with culture in a bioreactor.

The outer layer of hydrogel therefore forms the outer envelope of the microcompartment or of the capsule according to the invention, protecting its contents from the external environment, notably when the capsules are cultivated in a bioreactor. This is further enhanced by the addition of the layer or the mesh of hydrogel to the inner part of said microcompartment.

Advantageously, the layer or the mesh of hydrogel of the inner part is at least partially juxtaposed to the inner face of the outer layer.

According to a preferred object of the invention, the aforementioned advantages are further enhanced when the Young's modulus of the hydrogel of the inner part is between 0.01 and 200 kPa, more preferentially between 0.1 and 60 kPa, even more preferentially between 0.1 and 5 kPa. Preferentially, the Young's modulus of the hydrogel of the outer layer is greater than 10 kPa, more preferentially greater than 60 kPa, even more preferentially greater than 100 kPa.

Preferentially, the hydrogel of the layer or of the mesh of the inner part is entangled with the hydrogel of the outer layer, notably on the inner face of the outer layer. Also, the delimitation between the two hydrogels may not be perfectly clear despite their different Young's moduli. As a result, at least part of the hydrogel of the layer or of the mesh of the inner part can be entangled with the inner face of the outer layer.

Since the Young's modulus of the hydrogel of the inner part is strictly less than that of the hydrogel of the outer layer, the outer layer will be stiffer than the layer or the mesh of the inner part whose hydrogel is looser, slacker, notably due to the presence of a shorter chain, facilitating cell growth.

Preferentially, the hydrogel used is biocompatible, that is it is non-toxic to the cells. The hydrogel must allow the diffusion of oxygen and nutrients in order to supply the cells contained in the microcompartment and to enable them to survive. According to one particularly preferred embodiment, the outer layer of hydrogel comprises at least alginate. It may consist exclusively of alginate.

The alginate can be in particular a sodium alginate, composed of 80% α-L-guluronate and 20% β-D-mannuronate, having a Young's modulus greater than 10 kPa, preferentially greater than 60 kPa, more preferentially greater than 100 kPa.

According to another object, the alginate advantageously has an average molecular weight of 100 to 400 kDa, more preferentially the outer layer has a molecular weight of between 150 and 250 kDa. When the hydrogel of the outer layer is alginate, the concentration of the alginate solution intended to form said outer layer of the microcompartment is preferentially between 0.5 and 5% by mass, more preferentially the concentration is equal to 2% (plus or minus 0.5%) by mass.

When the concentration of the alginate solution intended to form the outer layer of the microcompartment is equal to 2%, the viscosity of the alginate is preferentially equal to 144 mPa/s.

Advantageously, the outer layer of hydrogel lacks cells.

The outer layer of hydrogel thus makes it possible to protect the cells from the external environment, to limit the uncontrolled proliferation of the cells, and their differentiation in case of differentiation.

According to a particularly preferred embodiment, the layer or the mesh of the inner part of hydrogel also comprises at least alginate. It may consist exclusively of alginate. The alginate may in particular be a sodium alginate, composed of 80% α-L-guluronate and 20% β-D-mannuronate, having a Young's modulus of between 0.01 kPa and 200 kPa, preferentially between 0.1 kPa and 60 kPa, more preferentially between 0.1 kPa and 5 kPa.

According to another object, the alginate has an average molecular weight of at most 75 kDa. When the hydrogel of the outer layer is alginate, the concentration of the alginate solution intended to form the outer layer of the microcompartment is preferentially between 0.25 and 2%, more preferentially between 0.25 and 1%, even more preferentially 0.5%. When the concentration of the alginate solution intended to form the outer layer of the microcompartment is 0.5%, the viscosity of the alginate is preferentially 3 mPa/s.

The layer or the mesh of the inner part having a low Young's modulus then has viscoelastic and viscoplastic properties that are particularly advantageous for obtaining an extracellular matrix substitute, in which cells are able to be housed and grow satisfactorily. Furthermore, alginate hydrogel is rheofluidic, that is its viscosity decreases as the shear rate increases, which is particularly advantageous when passing through the microfluidic injector. Finally, alginate having a low Young's modulus will also exhibit faster relaxation properties, also known as “fast-relaxing”, which allows cells to move, change shape, expand, proliferate and mechanically remodel the alginate-based matrix.

Also, the hydrogel of the outer layer and/or of the layer or of the mesh of the inner part is very preferentially alginate.

Despite the fact that mammalian cells are unable to interact with alginate, notably because it allows minimal protein adsorption, alginate can be used as an extracellular matrix substitute, such as Matrigel®. Alginate has the following advantages: alginate is well-characterized, easy to sterilize and to store, it can optionally be chemically modified, and offers interesting mechanical properties. Also, alginate is particularly suited to the context of the invention and provides good growth, low cell death and good amplification.

According to another preferred object of the invention, the cellular microcompartment comprises cells, an outer layer of hydrogel and a layer or a mesh in the inner part of hydrogel of lower molecular weight than the hydrogel of the outer layer.

Advantageously, the microcompartment according to any one of the preceding embodiments also comprises at least one layer of cells and/or at least one culture medium. When the microcompartment according to the invention comprises a layer or a mesh in the inner part of hydrogel, this is arranged between the outer layer of hydrogel and said cell layer. It is understood that the microcompartment can also comprise cells in suspension in the culture medium or optionally housed in the hydrogel.

According to one variant, the invention also relates to a cellular microcompartment comprising:
at least one layer of cells,
an outer layer of hydrogel, and
a layer or a mesh in the inner part of hydrogel arranged between the outer layer of hydrogel and said cell layer,
   characterized in that the inner part comprises a hydrogel with a molecular weight strictly less than that of the hydrogel of the outer layer.

Advantageously, the inventors observed better amplification, as well as the presence of cysts, mostly round in shape, at a concentration of 0.5%. At 2%, the cysts are substantially elongated in shape and the microcompartment has fewer cysts with respect to a lower alginate concentration. The higher the concentration of alginate, the greater the physical constraints in the capsule, making the gel tighter and, as a result, giving cells less room to multiply and move around. The cysts then take on a substantially elongated shape.

Thus, depending on the objective sought, or the cell type, the alginate constituting the layer or the mesh of the inner part of the microcompartment can be present at a total concentration of between 0.25 and 2% by mass, which makes it possible to obtain good amplification and good pluripotency, thus enabling its use in three-dimensional cell culture.

The layer or the mesh of the inner part of hydrogel, preferentially of alginate, may comprise other constituents, thus said layer or the mesh of the inner part of hydrogel preferentially comprises at least one peptide sequence, more preferentially a peptide sequence of interest capable of interacting with the cells notably making up the layer of cells present in the microcompartment according to the invention. For example, the peptide sequence may be a peptide or a protein.

According to a particularly preferred object, the peptide sequence is a YIGSR motif and/or an RGD motif. The YIGSR motif is a peptide derived from the β1 chain of laminin with a Tyrosine-Isoleucine-Glycine-Serine-Arginine sequence, facilitating cell adhesion to the matrix. The RGD motif is a peptide with an Arginine-Glycine-Asparagine sequence that also facilitates cell adhesion to the matrix.

Thus, in order to further improve the properties of this hydrogel-based extracellular matrix substitute, said layer or said mesh of the inner part of hydrogel comprises, preferentially, at least one peptide sequence, namely a YIGSR motif or an RGD motif. Efficiency is further enhanced when this YIGSR or RGD motif comprises a spacer, located between the motif of interest (YIGSR or RGD) and the hydrogel of the inner part. Preferentially, the spacer is a peptide sequence, said sequence comprising n glycine residues, thereby improving the accessibility to cells of the sequence of interest while enhancing the polymerization of the hydrogel of the inner part. More preferentially, the spacer comprises at least 3 glycine residues, preferentially between 3 and 30 glycine residues, even more preferentially between 6 and 15 glycine residues. Efficacy is particularly enhanced when the spacer comprises 12 glycine residues, in particular for cell amplification.

Also, according to another object, the invention also relates to a peptide sequence comprising a spacer consisting of n glycine (G) residues, and at least one YIGSR motif or at least one RGD motif. According to a preferred object, said peptide sequence is selected from the group consisting of SEQ ID NO:1, SEQ ID NO: 2, SEQ ID NO:3, SEQ ID NO:4, SEQ ID NO:5, and SEQ ID NO:6.

Thus, the hydrogel mesh of plant or synthetic origin of the cellular microcompartment according to the invention preferentially comprises at least one peptide sequence comprising n glycine residues, and at least one YIGSR motif or at least one RGD motif. The hydrogel mesh of plant or synthetic origin of the cellular microcompartment according to the invention is then functionalized with said peptide sequence comprising n glycine residues and at least said YIGSR motif or at least said RGD motif, making it possible to improve accessibility to the cells of the motif of interest while improving polymerization of the hydrogel of the inner part in order to improve cell survival and amplification.

According to one variant, such a YIGSR and/or RGD motif with or without a spacer, can also be used to functionalize the hydrogel of the outer layer and/or the hydrogel of the inner part.

According to another preferred object of the invention, the layer or the mesh of the inner part of hydrogel comprises at least a second hydrogel distinct from the first hydrogel of the layer or mesh of the inner part, more preferentially it is selected from fibrin, laminin, fibronectin, entactin, hyaluronic acid and collagen.

When the layer or the mesh of the inner part of hydrogel comprises fibrin, the fibrin is preferentially obtained from the polymerization of fibrinogen by a fibrinogen polymerization agent, advantageously said agent is thrombin, said agent can be added during encapsulation and/or after encapsulation. Also, the polymerization of the fibrinogen solution by the thrombin solution takes place during encapsulation and/or thereafter. When it takes place after encapsulation, the polymerization takes place within the newly formed drop or capsule, that is once the outer layer has stiffened.

The microcompartment is then a three-dimensional microcompartment, delimited by the outer layer of hydrogel and inside said outer layer, an inner part comprises the cells and the layer or the mesh of hydrogel. Advantageously, it comes in a variety of spherical shapes. Advantageously, the three-dimensional microcompartment is hollow, more preferentially, the hollow microcompartment is in the shape of an ovoid, a cylinder, a spheroid, a sphere or a teardrop.

For the purposes of the invention, “hollow microcompartment” means a three-dimensional microcompartment which has a cavity delimited by the outer layer of hydrogel, said cavity optionally comprising the layer or the mesh of hydrogel, culture medium and a plurality of cells, said cavity forming the inner part. By way of example, FIG. 8 describes a microcompartment comprising an outer layer of hydrogel, an inner part formed by the hollow cavity, the latter comprising a heterogeneous hydrogel mesh.

On the one hand, the outer layer of hydrogel protects the cells from the external environment, limiting uncontrolled cell proliferation and differentiation in the event of differentiation; on the other hand, the layer or mesh in the inner part of hydrogel provides a suitable environment for cell growth and multiplication thereof. Cell proliferation can optionally be controlled based on the concentration of the hydrogel as described previously.

In the context of the invention, the cells present in the microcompartment can be any type of cell, in particular the cells are eukaryotic cells. More preferentially, the cells are human or plant or animal cells.

In a particular embodiment, the microcompartment comprises pluripotent stem cells. A pluripotent stem cell, or pluripotent cell, refers to a cell that has the ability to form all the tissues present in the whole organism of origin, without being able to form an entire organism as such. The pluripotent stem cells can in particular be induced pluripotent stem cells (iPS), MUSE cells (“Multilineage-differentiating Stress Enduring”) that are found in skin and bone marrow of adult mammals, or embryonic stem cells (ES). According to one embodiment, the microcompartment according to the invention does not comprise embryonic stem cells (ES).

According to a particularly suitable variant of the invention, the microcompartment according to the invention comprises human or animal induced pluripotent stem cells.

In another particular embodiment, the microcompartment according to the invention comprises human or animal multipotent cells and/or human or animal progenitor cells derived from these multipotent cells and/or cells which are undergoing differentiation. The multipotent and/or progenitor cells were preferentially obtained from pluripotent stem cells, in particular human pluripotent stem cells, or optionally from non-pluripotent human cells, the transcriptional profile of which was artificially modified to match that of specific multipotent and/or progenitor cells, typically by forced expression of specific transcription factors for the target cellular phenotype. Preferentially, the multipotent and/or progenitor cells were obtained from pluripotent stem cells after being brought into contact with a solution capable of initiating the differentiation of said stem cells.

According to another variant, the microcompartment according to the invention comprises human or animal differentiated cells. The differentiated cells were preferentially obtained from pluripotent stem cells or progenitor cells, in particular human pluripotent stem cells or human progenitor cells, or optionally from non-pluripotent human cells whose transcriptional profile was artificially modified to join that of particular differentiated cells, typically by forced expression of specific transcription factors of the target cell phenotype. Preferentially, the differentiated cells were obtained from pluripotent or multipotent or progenitor stem cells after being brought into contact with a solution capable of initiating differentiation of said stem cells. According to one variant, the cellular content of the microcompartment comprises homogeneous or mixed cell identities.

The differentiated cells can in particular be in the form of at least one layer of cells or in the form of a three-dimensional tissue, a cell aggregate or a microtissue or in the form of a plurality of tissues or microtissues in the microcompartment. It may be a compacted or non-compacted tissue or micro-tissue, with or without a lumen.

The microcompartment according to the invention may therefore comprise several types of cells. In particular, the microcompartment according to the invention may comprise for example stem cells induced to pluripotency and/or multipotent cells and/or progenitor cells and/or in the process of differentiation and/or differentiated cells.

According to a particular object of the invention, the microcompartment according to the invention is obtained after a plurality of cell division cycles. Indeed, the cells included in the microcompartment according to the invention are cells obtained by amplification, from at least one cell.

Also, the cells present in the microcompartment according to the invention were obtained after at least two cell division cycles after encapsulation in an outer layer of hydrogel of at least one cell.

Preferably, the cells present in the microcompartment according to the invention were obtained after at least 2, 3, 4, 5, 6, 7, 8, 9, 10, 11, 12, 13, 14, 15, 20, 25, 28, 30 cell division cycles after encapsulation in an outer layer of hydrogel of at least 1 cell, preferentially between 1 and 5, between 1 and 10, between 1 and 15, between 1 and 20, between 1 and 30, between 1 and 40, between 1 and 50, between 1 and 60, between 1 and 100 cells. For example, the cells present in the microcompartment were obtained after at least six cell division cycles after the encapsulation in an outer layer of hydrogel of at least 1 cell, preferentially between 1 and 50 cells.

Preferentially, the microcompartment is obtained after at least 2 passes after encapsulation, more preferentially at least 3, 4, 5, 6, 7, 8, 9 or 10 passes. Each pass can last for example at least 1 day, or between 2 and 50 days, in particular between 3 and 10 days.

Preferentially, all of the cells initially encapsulated in the microcompartment before the first cell division cycle represents a volume less than 50% of the volume of the microcompartment in which they are encapsulated, more preferentially less than 40%, 30%, 20%, 10% of the volume of the microcompartment in which they are encapsulated.

Thus, according to one embodiment, the cells present in the microcompartment according to the invention were obtained after at least 2, 3, 4, 5, 6, 7, 8, 9, 10, 11, 12, 13, 14, 15, 20, 25, 28, 30 cell division cycles, after the encapsulation in an outer layer of hydrogel of cell(s) representing a volume less than 50% of the volume of the microcompartment in which they are encapsulated, more preferentially less than 40%, 30%, 20%, 10% of the volume of the microcompartment in which they are encapsulated.

Preferentially, in the microcompartment according to the invention, the cells represent more than 50% by volume with respect to the volume of the microcompartment, even more preferentially more than 60%, 70%, 75%, 80%, 85%, 90% by volume with respect to the volume of the microcompartment.

The microcompartment according to the invention comprises several cells, preferentially at least 20 cells, even more preferentially at least 100, at least 500, at least 1000, at least 10,000.

According to another preferred object of the invention, the capsule is obtained by encapsulation by means of a co-injection carried out concentrically via a microfluidic injector forming a jet at the injector outlet consisting of the mixture of the various useful solutions, said jet breaking up into drops. The drops are then collected in a calcium bath capable of stiffening the hydrogel solution to form the outer layer of each microcompartment.

According to a variant allowing the formation of a tube, the co-injection is also carried out concentrically via a microfluidic injector, said injector comprising a tip, said tip being in contact with a calcium solution, forming a jet at the injector outlet consisting of the mixture of said solutions, said jet forming the tube in the calcium solution.

The microcompartment according to the invention may also comprise other elements, in particular a culture medium. The culture medium is a medium suitable for the cells present in the microcompartment according to the knowledge of a skilled person.

According to another preferred object of the invention, the microcompartment comprises at least one lumen. Said lumen may contain a liquid, notably the culture medium and/or a liquid secreted by the cells. Advantageously, the presence of this hollow part enables the cells to have a small diffusive volume of which they can control the composition, promoting cellular communication. The three-dimensional arrangement in a single layer or spherical cellular base surrounding the lumen or the central lumen may also be called a cyst.

The lumen is preferentially generated, at the time of the formation of the cyst, by the cells which multiply and develop within the hydrogel constituting the layer or the mesh in the inner part of the cellular microcompartment.

According to another preferred object, the layer of cells, the layer or the mesh of the inner part of hydrogel and the outer layer are organized around the lumen, more preferentially they are organized successively around the lumen.

The cyst-shaped conformation it makes it possible to reduce the pressures experienced by the stem cells relative to the 2D cultures or aggregates. This configuration also makes it possible to reduce cell mortality and to increase the culture amplification factor. As a result, this makes it possible to reduce the number of passes and dissociations required, to reduce the culture time necessary to reach the final required number of cells.

According to one embodiment, the microcompartment may comprise a plurality of cysts or tissues or microtissues.

The cellular microcompartment according to the invention is preferentially closed or partially closed, that is the outer layer is closed or partially closed. More preferentially, the microcompartment is closed.

The microcompartment according to the invention can be in any three-dimensional form, that is, it may have the shape of any object in space. The microcompartment may have any form compatible with cell encapsulation. Preferentially, the microcompartment according to the invention is in a spherical or elongated shape. It may have the shape of an ovoid, a cylinder, a spheroid or a sphere. It may in particular be in the form of a hollow spheroid, a hollow ovoid, a hollow cylinder or a hollow sphere.

It is the outer layer of the microcompartment, that is the hydrogel layer, which imparts its size and shape to the microcompartment according to the invention. Preferentially, the smallest dimension of the microcompartment according to the invention is between 10 μm and 1 mm, preferentially between 100 μm and 700 μm. It may be between 200 μm and 600 μm, in particular between 300 μm and 500 μm.

Its largest dimension is preferentially greater than 10 μm, more preferentially between 10 μm and 1 m, even more preferentially between 10 μm and 50 cm.

The microcompartment according to the invention may optionally be frozen to be stored. It will then have to be thawed before it is used.

The invention also relates to a plurality of microcompartments together. Also, the invention also relates to an assembly or a series of cellular microcompartments as described previously comprising at least one cellular microcompartment according to the invention.

The invention also relates to an assembly or a series of microcompartments of at least two three-dimensional cellular microcompartments, each microcompartment comprising at least one outer layer of hydrogel and inside said outer layer at least one layer or mesh of hydrogel and at least one cell, wherein at least one microcompartment is a microcompartment according to the invention.

The assembly of microcompartments according to the invention preferentially comprises between 2 and 1016 microcompartments.

Preferably, the series of microcompartments according to the invention is in a culture medium, in particular in an at least partially convective culture medium.

According to a particularly suitable embodiment, the object of the invention is an assembly of cellular microcompartments in a closed chamber, such as a bioreactor, preferentially in a culture medium in a closed chamber, such as a bioreactor.

The presence of an outer layer of hydrogel and optionally an intermediate layer of isotonic aqueous solution enables a uniform distribution of the cells between the microcompartments. Moreover, this hydrogel layer makes it possible to prevent microcompartments from merging, these merger events being a major source of variability which is unfavorable for phenotypic homogeneity of cells.

Thus, the microcompartment according to any one of the previously described embodiments or the assembly of microcompartments according to any one of the previously described embodiments, is also particularly suitable for use in cell culture, in particular in three-dimensional cell culture. These enable the production of cells of interest in large quantities, or even organoids of interest, suitable for use, for example, in cell therapy. Also, the invention also relates to a microcompartment according to the invention or a microcompartment assembly according to the invention, for use as a medicament.

Thus, the microcompartment is particularly suitable for use in a clinical setting.

According to another object, the invention relates to the use of the microcompartment according to any one of the preceding objects, for the production of cells, microtissues, tissues, preferentially for the large-scale production of such cells and/or tissues.

The microcompartment according to the invention can also be used for the production of animal or plant cells for human or animal food consumption. This use is particularly useful for creating substitutes for meat products such as meat, in order to limit the consumption of meat products.

### Preparation Method

The microcompartment can be obtained by different means known to a skilled person to prepare microcompartments or capsules.

According to another aspect of the invention, a method for preparing microcompartments according to the invention has also been developed by the inventors.

Thus, a particularly suitable method for preparing a microcompartment according to the invention comprises the following steps:
a. mixing cells, optionally previously incubated in a culture medium,
b. encapsulating the mixture from step (a) in an outer layer of hydrogel;
c. cultivating the capsules obtained in step (b) in a culture medium, preferentially in a bioreactor, preferentially for at least 1 day, preferentially from 3 to 50 days, and
d. optionally recovering the cellular microcompartments obtained, characterized in that, during step (a) and/or during step (b), a hydrogel solution of plant or synthetic origin is added, the Young's modulus of which is strictly less than the Young's modulus of the hydrogel used to form the outer layer of hydrogel.

Advantageously, the method according to the invention may comprise additional steps. Thus, preferentially, the cells are incubated prior to the step of mixing the cells with a hydrogel solution having a Young's modulus less than that of the hydrogel of the outer layer (ii) in a suitable culture medium. Said culture medium preferentially comprises at least one cytoprotective factor, more preferentially at least one inhibitor of apoptosis.

The inhibitor of apoptosis may for example be one or more inhibitor(s) of RHO/ROCK (RHO-associated protein kinase) pathways, or any other inhibitor of apoptosis known to a skilled person. The inhibitor of apoptosis must make it possible to promote cell survival, the adhesion of the cells to fibrin during the formation of the outer layer of hydrogel.

The method according to the invention may comprise a step of dissociation of the cells by chemical, enzymatic or mechanical dissociation, prior to or simultaneously implemented in the cell incubation step, itself carried out prior to mixing step a). This step is particularly important in the case of adherent cells.

The encapsulated cells are suspended in the form of single cells and/or cell clusters. Preferably, the single cells represent less than 50% by number of the totality of the encapsulated cells, more preferentially the single cells are hPSC cells. Indeed, it is preferable to encapsulate clusters of cells because this reduces the appearance of the mutagenesis phenomenon.

Preferentially, the encapsulation step b) comprises the following sub-steps:
bringing the mixture from step (a) into contact with a hydrogel solution intended to form the outer layer (i) to form at least one drop, and
collecting the drops in a calcium bath capable of stiffening said hydrogel solution to form the outer layer of each microcompartment.

Once the outer hydrogel layer has been stiffened by the calcium bath, the microcompartment is formed. This can then be rinsed off. Advantageously, the Young's modulus of the hydrogel in the inner part, which is less than that of the hydrogel in the outer layer, prevents/limits stiffening of the hydrogel in the inner part by the calcium bath. In particular, given that the Young's modulus of the hydrogel in the inner part is strictly less than that of the hydrogel in the outer layer, the stiffening process using the calcium bath does not allow the hydrogel in the inner part to stiffen during the time the capsules are in use and therefore does not facilitate cell multiplication.

Preferentially, step b) is carried out by simultaneous co-injection of the hydrogel solution intended to form the outer layer (i), of the mixture from step a) optionally comprising the hydrogel solution with a Young's modulus strictly less than that of the hydrogel of the outer layer (ii), and optionally of an intermediate solution (iii) optionally comprising the hydrogel solution with a Young's modulus strictly less than that of the hydrogel of the outer layer (ii); said co-injection is carried out concentrically via a microfluidic or millifluidic injector forming a jet at the injector outlet made up of the mixture of said solutions, said jet breaking up into drops.

When the intermediate solution (iii) comprises the hydrogel solution with a Young's modulus less than that of the hydrogel of the outer layer (ii), said hydrogel solution (ii) is not present in the mixture of step a).

According to a preferred object of the invention, the isotonic intermediate solution is a sorbitol solution.

According to one object of the invention, the final opening diameter of the microfluidic injector is between 50 and 800 μm, preferentially between 80 and 240 μm, and the flow rate of each of the solutions is between 0.1 and 2000 mL/h, preferentially between 10 and 2000 mL/h, more preferentially between 11 and 100 mL/h.

According to a variant, step b) is carried out by simultaneous co-injection of the hydrogel solution intended to form the outer layer (i), of the mixture from step a) optionally comprising the hydrogel solution with a Young's modulus strictly less than that of the hydrogel of the outer layer (ii), and optionally of an intermediate solution (iii) optionally comprising the hydrogel solution with a Young's modulus strictly less than that of the hydrogel of the outer layer (ii); said co-injection is carried out concentrically via a microfluidic or millifluidic injector, said injector comprising a tip, said tip being in contact with a calcium solution, forming a jet at the injector outlet consisting of the mixture of said solutions, said jet forming a tube.

When the injector tip is in contact with the calcium solution, the final opening diameter of the microfluidic injector is preferentially between 50 and 1000 μm, more preferentially between 80 and 300 μm, and the flow rate of each solution is between 1 and 100 mL/h.

According to another variant of the invention, the inner part comprising the layer or the mesh of hydrogel also comprises at least a second hydrogel distinct from the first hydrogel constituting the layer or the mesh of the inner part, more preferentially fibrin. Fibrin is preferentially obtained from the polymerization of fibrinogen by a fibrinogen polymerization agent, advantageously said agent is thrombin, said agent can be added during encapsulation and/or after encapsulation.

Also, the thrombin solution is co-injected with the other solutions, preferentially mixed with the isotonic intermediate solution and the fibrinogen solution is mixed with the mixture from step a).

Preferentially, the fibrinogen concentration is between 10 and 25 mg/mL, preferentially 14-20 mg/mL, more preferentially between 20 mg/mL.

According to another object of the invention, the concentration of thrombin is preferentially between 0.001 U/mL and 2 U/mL, more preferentially between 0.01 U/mL and 1 U/mL, between 0.01 U/mL and 0.05 U/mL, even more preferentially 0.04 U/mL. “U” refers to a unit of enzyme activity (that is the concentration of an enzyme), which represents the amount of enzyme required to process one micromole of substrate in 1 minute. It is understood that the indicated concentration is the one in the mixture. Indeed, advantageously thrombin is mixed with the other constituents according to a 1:1 ratio. Also, within the capsule, when the concentration of thrombin, before mixing, is 0.01 U/mL, the concentration in the capsule is about 0.01 U/mL.

The steps subsequent to the encapsulation can be carried out with or without stirring. Preferentially, the steps subsequent to the encapsulation are carried out under permanent or sequential stirring. This stirring is important because it maintains the homogeneity of the culture environment and avoids the formation of any diffusion gradient. For example, it allows homogeneous control of cellular oxygenation level; thus avoiding phenomena of hypoxia-related necrosis or hyperoxia-related oxidative stress. Therefore, it avoids an increase in cell mortality and/or oxidative stress.

Preferentially, after the step of culturing the obtained capsules, the method comprises a step which consists of rinsing the capsules resulting from step (d), advantageously so as to eliminate the cytoprotective factor, such as the inhibitor of apoptosis.

When, the method according to the invention comprises a step of rinsing the capsules obtained, the solution constituting the calcium bath is removed and replaced by a suitable medium for culturing the microcompartments according to the invention, preferentially an isotonic solution, more preferentially a culture medium containing an inhibitor of apoptosis. The rinsing step of the capsules obtained is carried out after step d).

In a preferred variant, the method according to the invention comprises at least one re-encapsulation of the cells after step (d), preferentially after the rinsing step if such a step is present after step d). “At least one re-encapsulation of the cells” means at least two encapsulation cycles. Preferentially, each encapsulation cycle corresponds to a pass. In this variant of the method (at least one re-encapsulation of the cells after step (d)), the number of cell divisions of the entire method (for all the passes) is at least 3, 4, 5, 6, 7, 8, 9, 10, 11, 12, 13, 14, 15, 20, 25, 30 cell division cycles.

In a method according to the invention, there may be several re-encapsulations, preferentially between 1 and 100, notably between 1 and 10 re-encapsulation(s).

Each re-encapsulation can comprise:
a. step which consists of dissociating the microcompartment or the series of microcompartments in order to obtain a suspension of cells or a suspension of cell clusters; the outer layer of hydrogel may be eliminated notably by hydrolysis, dissolution, piercing and/or breaking by any biocompatible means, that is means which are not toxic to the cells. For example, the elimination may be accomplished using phosphate-buffered saline, a divalent ion chelator, an enzyme such as alginate lyase if the hydrogel comprises alginate, and/or laser microdissection, and
b. a step of re-encapsulating all or part of the cells or cell clusters in a hydrogel capsule.

Re-encapsulation is a means suitable for increasing the cell amplification resulting from the pluripotent step, and reducing the risks of mutation.

According to a particular embodiment, the re-encapsulation comprises the following steps:
a. eliminating the outer layer of hydrogel,
b. resuspending the cells that were contained in the microcompartment so as to obtain single cells and/or at least one assembly or cluster of cells in an isotonic medium, preferentially a culture medium containing an inhibitor of apoptosis,
c. encapsulating the suspension of cells in a hydrogel layer;
d. preferentially, cultivating the microcompartments obtained in an isotonic solution containing an inhibitor of apoptosis, preferentially a culture medium containing an inhibitor of apoptosis;
e. preferentially, rinsing the microcompartments, advantageously, so as to eliminate the inhibitor of apoptosis;
f. cultivating the microcompartments in an isotonic solution, preferentially a culture medium, for at least one cell division cycle, and
g. optionally recovering the cellular microcompartments obtained.

### Kit According to the Invention

The invention also relates to a kit, said kit comprising a hydrogel solution with a Young's modulus strictly less than that of the hydrogel of the outer layer (i) and a hydrogel solution with a Young's modulus greater than that of the hydrogel of the layer or of the mesh of the inner part intended to form the outer layer (ii), optionally an intermediate solution (iii).

According to another aspect, the invention relates to the use of said kit for preparing a microcompartment according to the invention, said kit comprising at least one hydrogel solution intended to form the outer layer of the microcompartment (ii) and a hydrogel solution of plant or synthetic origin whose Young's modulus is strictly less than that used to form the outer layer (i), and optionally an intermediate solution (iii).

Preferentially, said kit comprises a low-molecular-weight hydrogel solution (i) and a high-molecular-weight hydrogel solution (ii), and optionally an intermediate solution (iii), in particular the low-molecular-weight hydrogel is less than that of the high-molecular-weight hydrogel.

According to another object, the invention also relates to a kit comprising at least one alginate solution of at most 75 kDa, an alginate solution between 150 and 250 kDa, an isotonic solution, preferentially a sorbitol solution, a calcium solution, a suitable culture medium. According to one variant, said kit is a kit-of-parts.

The invention is now showed by non-limiting examples of compositions according to the invention and by results.

### EXAMPLES

### Example 1—Capsule According to One Embodiment of the Invention

This example describes a particular embodiment of the invention, also shown in FIG. 2, wherein the 0.5% alginate solution, intended to form the inner alginate layer as a matrix substitute, is added to the sorbitol solution and co-injected via the microfluidic injector with the mixture of cells in the culture medium, and with the 2% alginate solution, intended to form the outer alginate layer.

The microfluidic injector enabling the co-injection of the various solutions comprises three lines upstream of the nozzle. The solution containing sorbitol and 0.5% alginate is injected in line “IS”. The 2% alginate solution and the cell suspension in the culture medium are injected in line “A” and line “CS” respectively, and then the encapsulation is carried out.

Once the encapsulation has been carried out, the drops are collected in the CaCl2 bath allowing the stiffening of the 2% alginate and the formation of the alginate shell forming the microcompartment or capsule. This CaCl2 solution comprising the capsules is then rinsed with a serum-free cell culture medium. The capsule then has the following characteristics: an outer alginate layer having a Young's modulus of the order of 120 kPa and an inner alginate layer (matrix substitute) having a Young's modulus of the order of 1 kPa.

### Example 2—Capsule According to One Embodiment of the Invention

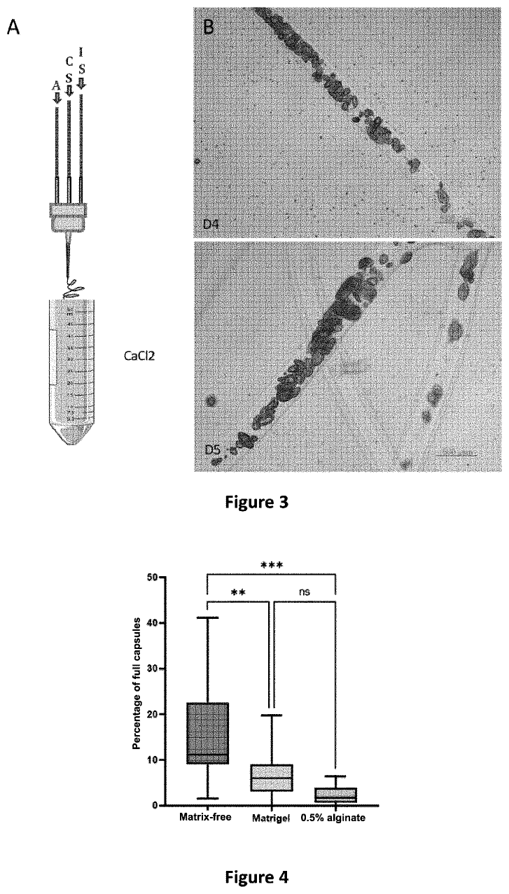

This example describes a particular embodiment of the invention, also shown in FIG. 3, wherein the 0.5% alginate solution intended to form the inner alginate layer as a matrix substitute is added to the sorbitol solution and co-injected via the microfluidic injector with the mixture of cells in the culture medium, and with the 2% alginate solution intended to form the outer alginate layer.

The microfluidic injector enabling the co-injection of the various solutions comprises three lines upstream of the nozzle. The solution containing sorbitol and 0.5% alginate is injected in line “IS”. The 2% alginate solution and the cell suspension in the culture medium are injected in line “A” and line “CS” respectively. In this embodiment, the tip of the microfluidic injector is brought into contact with the calcium solution allowing encapsulation in the form of a tube.

Once the encapsulation has been carried out, the tubes are collected in the CaCl2 bath allowing the stiffening of the 2% alginate and the formation of the alginate wall forming the tube. This CaCl2 solution comprising the tubes is then rinsed with a serum-free cell culture medium. The tube then has the following characteristics: an outer alginate wall having a Young's modulus of the order of 120 kPa and an inner alginate layer (matrix substitute) having a Young's modulus of the order of 1 kPa.

### Example 3—Measuring the Viscosity of Capsules According to the Invention and Outside the Scope of the Invention

In the context of this test, the inventors used an iPS cell line that was generated according to the usual standards of two-dimensional iPS culture, then the cells were detached from the flasks via the action of an enzyme, according to the knowledge of a skilled person, and transferred to the culture medium suitable for the culture of iPS.

The iPS cells were mixed in a suitable culture medium so as to obtain a cell density of around 1.5 M/mL. The 0.5% alginate solution intended to form the inner alginate layer as a matrix substitute was mixed with a sorbitol solution. A 2% alginate solution intended to form the outer alginate layer was also prepared. The various solutions were then loaded via the dedicated lines and co-injected simultaneously by means of a microfluidic injector. The amount of cells encapsulated is on the order of 0.6*10\hat{ }6.

The same protocol was used in order to obtain capsules without an exogenous extracellular matrix and capsules with Matrigel®, with the difference that the sorbitol solution is injected alone and the 0.5% alginate solution is replaced by a Matrigel® solution.

From D1 to D5 after encapsulation, the capsules are visually checked. On D5, the appearance of the cells, the amount of cells, their viability and pluripotency is also observed.

The viscosity of the constituents of each microcompartment was measured using a rolling-ball viscometer, measuring the rolling time of a ball through opaque and transparent liquids according to the Hôppler falling ball principle. The results show the viscosity of each constituent present in each type of microcompartment comprising respectively:
a Matrigel®-like extracellular matrix (outside the scope of the invention),
an absence of an exogenous extracellular matrix or extracellular matrix substitute, that is only the culture medium (outside the scope of the invention),
an alginate-like extracellular matrix (invention).

The results are presented in Table 1 below.

TABLE 1

| Capsules/Matrices | Viscosity (at a shear rate = 60 s\hat{ }−1) | |
|---|---|---|
| Cell culture medium | 1.31 | Po |
| Matrigel ® at 4° C. | 22 | Po |
| 0.5% alginate | 3.0961 | Po |

The capsules comprising only cell culture medium, that is without an exogenous extracellular matrix, have the lowest viscosity, while 0.5% alginate-based capsules have a viscosity 2.3 times higher. The Matrigel®-based capsules have the highest viscosity. However, Matrigel® is a highly complex medium, with wide variations in viscosity within the matrix. Locally, viscosity can be very high or very low, which implies a very heterogeneous viscosity within Matrigel® and therefore a high degree of heterogeneity in the formation of cellular aggregates within Matrigel®. Conversely, alginate has a viscosity fairly close to that of the culture medium, promoting cell growth. Furthermore, the viscosity is very homogeneous resulting in good cell distribution within the alginate-based capsules.

### Example 4—Measuring the Percentage of Empty Capsules

This test compares the seeding rate of capsules comprising only culture medium, Matrigel® or 0.5% alginate (invention). The capsules are prepared according to the method described in Example 3 and the empty capsule percentage is measured on DO using the following method. 10 photos are taken for each condition. Using software such as ImageJ, each photo is analyzed in order to count the empty and full capsules (at least one cell in the capsule), then the software determines the ratio between the two and multiplies this by 100 to obtain a percentage.

The results are shown in FIG. 4. The inventors observed better cell seeding in capsules with 0.5% alginate with respect to Matrigel® capsules and capsules comprising only the cell culture medium. As this alginate-based matrix is more viscous, and the viscosity more homogeneous, it enables the cells to remain in suspension and thus to distribute themselves uniformly inside the capsule during the process. This is notably in contrast to capsules comprising Matrigel®, a matrix having highly heterogeneous viscosity. Thus, few capsules according to the invention are devoid of cells, demonstrating a better capacity for cell seeding in capsules according to the invention and therefore a greater proportion of capsules comprising cells and therefore subsequently cysts, allowing their subsequent use, notably in cell therapy.

### Example 5—Measuring Capsule Size and the Number of Cysts Per Capsule

This test compares capsules comprising only culture medium, Matrigel® or 0.5% alginate (invention). The capsules are prepared according to the method described in Example 3. The size of the capsules is measured on DO, for example using ImageJ software comprising an area selection tool, and the amount of cysts per capsule is measured on DO according to the method described in Example 4.

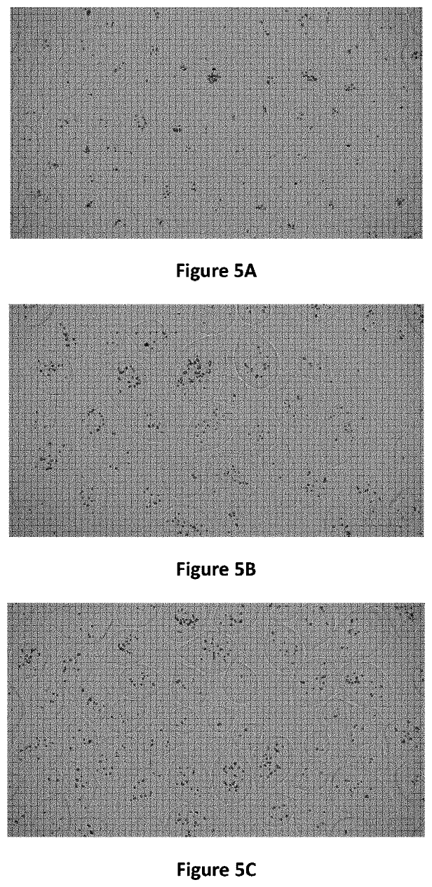

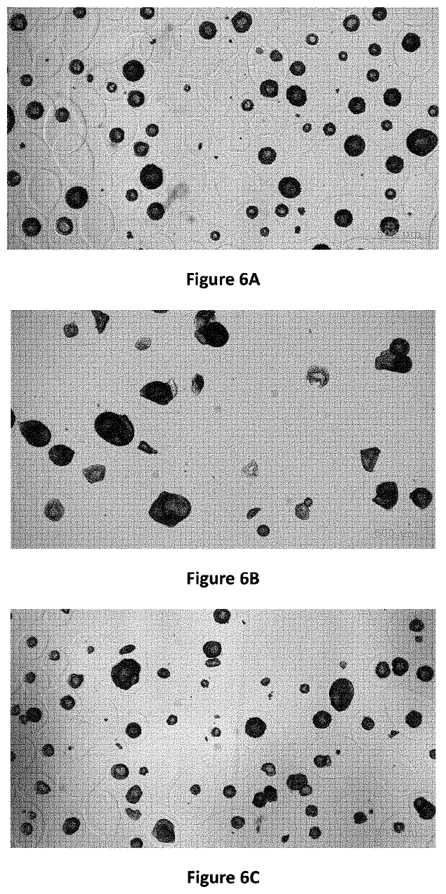

The results are presented in Table 2 below and in FIG. 5a, FIG. 5b, FIG. 5c, FIG. 6a, FIG. 6b, FIG. 6c,

TABLE 2

| | Average capsule | Average number of cysts |
|---|---|---|
| Capsules/matrix | size in μm2 | detected/Capsules |
| Cell culture medium | 359 | 2.43 |
| Matrigel ® at 4° C. | 440 | 7.27 |
| 0.5% alginate | 429 | 5.25 |

After 5 days of culture, dead cells can be observed inside some capsules when cultivated without a matrix, making the outer layer of the cyst somewhat irregular. In Matrigel®-based capsules, cells grow rapidly and more or less each capsule is filled with an aggregate, several aggregates can also be observed in the same capsules and they tend to merge to form a larger aggregate. The outer layer of the cyst is smooth and free of dead cells. In the 0.5% alginate-based capsules, the inventors also observed several aggregates in the same capsules, fewer dead cells with respect to the matrix-free condition but a smaller structure with respect to Matrigel®. The lumen is still visible after 5 days of culture. The average size of Matrigel®-based and 0.5% alginate-based capsules is similar allowing cells to multiply properly. Finally, the inventors observed a greater number of cysts in alginate-based capsules than in cell medium-based capsules.

### Example 6—Measuring Amplification and Pluripotency after 5 Days in Culture

This test compares capsules comprising only culture medium, Matrigel®-based capsules or 0.5% alginate-based capsules (invention). The capsules are prepared according to the method described in Example 3. The amplification and the pluripotency of the cells present in each type of capsule is measured on D5 according to the following method. The ratio of the number of cells encapsulated on DO to the number of cells obtained (and alive) on D5 is determined. On D5, the capsules are dissolved and the aggregates dissociated, then the cells are counted.

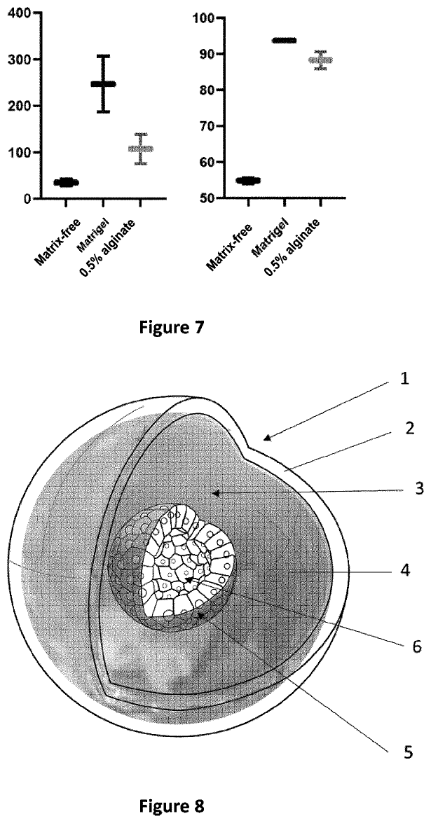

The results are shown in FIG. 7.

On D7, the inventors observed better amplification of cells cultivated in 0.5% alginate-based capsules compared with cells cultivated in matrix-free capsules. In Matrigel®-based capsules, the amplification factor is higher than in the other two conditions but the variability between experiments is also much higher, which is incompatible with the clinical use of this technology.

With regard to pluripotency, on D7, the inventors also observed a lower average percentage of Oct4/Nanog-positive cells in the 0.5% alginate with respect to the Matrigel® condition, but higher with respect to the matrix-free condition. Experiment-to-experiment variability is also reduced with 0.5% alginate.

As a result, the capsules according to the invention protect cells and aggregates from the mechanical stresses of culture in a bioreactor, with the contribution of the protective shell on the one hand, but also with the hydrogel as a matrix substitute in the capsule that provides an additional protective mesh for the cells and aggregates. This mesh is loose enough to allow migration and amplification of cells while maintaining their pluripotent character.

### Example 7—Measuring Amplification and Pluripotency after 5 Days of Culture in a Cellular Microcompartment Comprising Alginate Functionalized with a YIGSR Motif

The aim of this test is to compare 0.5% alginate-based capsules functionalized with a YIGSR motif and a spacer comprising 6, 9 and 12 glycine residues respectively (SEQ ID NO:1; SEQ ID NO:2; SEQ ID NO:3). The capsules are prepared according to the method described in Example 3. The amplification and the pluripotency of the cells present in each type of capsule is measured on D5 according to the following method. The ratio of the number of cells encapsulated on DO to the number of cells obtained (and alive) on D5 is determined (Factor-X). On D5, the capsules are dissolved and the aggregates dissociated, then the cells are counted.

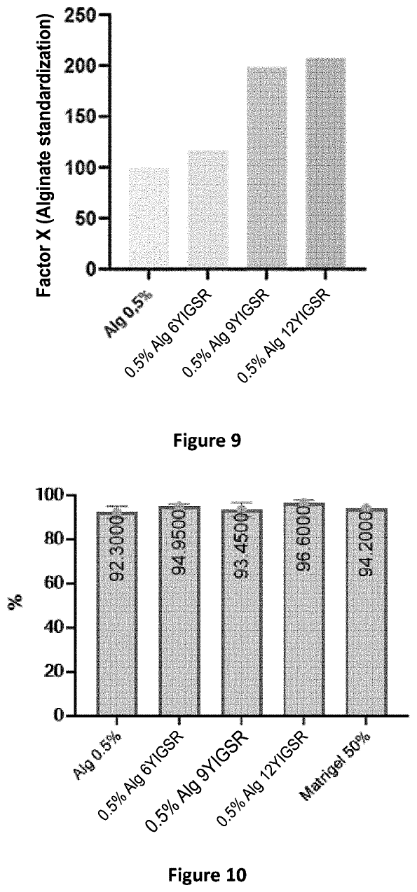

The results of the amplification and the pluripotency are shown in FIG. 9 and FIG. 10 respectively,

The inventors then found that functionalizing alginate (matrix substitute) with a YIGSR motif improved cell adhesion to the alginate-based matrix substitute, therefore enhancing the survival of said cells and thus the amplification of said cells. This effect is further enhanced when the YIGSR motif also comprises a spacer of 6, 9 and 12 glycine residues respectively (SEQ ID NO:1; SEQ ID NO:2; SEQ ID NO:3).

Finally, the inventors also found that functionalization with the YIGSR motif, optionally comprising a spacer, maintained pluripotency under every condition.

### Example 8—Measuring Amplification after 5 Days of Culture in a Microcompartment According to the Invention Comprising Alginate as a Matrix Substitute and Differentiated Cells of the Endoderm and Mesoderm

The aim of this test is to demonstrate the efficacy of the 0.5% alginate-based microcompartment comprising differentiated cells, namely cells of the endoderm and mesoderm. The capsules are prepared according to the method described in Example 3. The capsules containing iPSCs are cultivated for 4 days. Said cells were then differentiated for 5 days, by adding media allowing the differentiation of iPSC cells into endodermal and also mesodermal sheets.

The amplification of cells present in each type of capsule is measured on D5 according to the following method. The ratio of the number of cells encapsulated on DO to the number of cells obtained (and alive) on D5 is determined (Amplification factor). On D5, the capsules are dissolved and the aggregates dissociated, then the cells are counted.

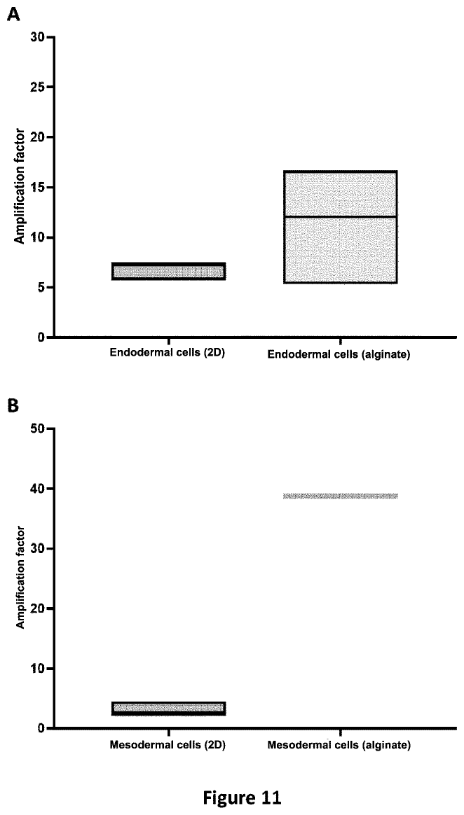

The amplification results are shown respectively in FIG. 11.

The inventors then observed a better amplification of cells cultivated in 0.5% alginate-based capsules compared with cells cultivated in two dimensions, thus demonstrating the capacity for amplification and culture of differentiated cells, notably cells of the endoderm and mesoderm in a microcompartment according to the invention.

1. A three-dimensional cellular microcompartment comprising:
an outer layer of hydrogel, and
an inner part comprising at least one cell and at least one layer or a mesh of hydrogel of plant or synthetic origin,
characterized in that the Young's modulus of the hydrogel in the inner part is strictly less than the Young's modulus of the hydrogel in the outer layer.

2. The cellular microcompartment according to claim 1, characterized in that the layer or the mesh of hydrogel of the inner part is at least partially juxtaposed to the inner face of the outer layer.

3. The cellular microcompartment according to claim 1, characterized in that the Young's modulus of the hydrogel in the inner part is between 0.01 and 200 kPa, preferentially between 0.1 and 60 kPa.

4. The cellular microcompartment according to claim 1, characterized in that the Young's modulus of the hydrogel in the inner part is between 0.1 and 5 kPa.

5. The cellular microcompartment according to claim 4, characterized in that the Young's modulus of the hydrogel of the outer layer is greater than 10 kPa, preferentially greater than 60 kPa.

6. The cellular microcompartment according to claim 1, characterized in that the hydrogel in the outer layer and/or in the inner part is alginate or comprises at least alginate.

7. The cellular microcompartment according to claim 1, characterized in that the inner part also comprises at least one culture medium.

8. The cellular microcompartment according to claim 1 characterized in that the inner part comprises at least one layer of cells.

9. The cellular microcompartment according to claim 8 characterized in that the layer or the mesh of hydrogel of the inner part is arranged between the outer layer of hydrogel and said at least one cell layer.

10. The cellular microcompartment according to claim 1, characterized in that the layer or mesh of hydrogel of the inner part comprises at least one peptide sequence.

11. The cellular microcompartment according to claim 1, characterized in that the layer or mesh of hydrogel of the inner part, in addition to the hydrogel of plant or synthetic origin, comprises at least one other hydrogel selected from fibrin, collagen, fibronectin, entactin, hyaluronic acid and laminin.

12. The cellular microcompartment according to claim 1, characterized in that the microcompartment is closed.

13. The microcompartment according to claim 1, characterized in that the microcompartment has the shape of an ovoid, a cylinder, a spheroid, a sphere or a teardrop.

14. The cellular microcompartment according to claim 1, characterized in that the cell or the cells present in the inner part are selected from human, animal and plant eukaryotic cells.

15. The cellular microcompartment according to claim 1, characterized in that the cell or the cells present in the inner part are pluripotent cells and/or progenitor cells and/or differentiating cells and/or differentiated cells.

16. The cellular microcompartment according to claim 1, characterized in that the inner part of the microcompartment comprises at least one layer of cells and at least one lumen, and in that the layer of cells, the layer or mesh of hydrogel of the inner part and the outer layer are successively organized around said lumen.

17. The cellular microcompartment according to claim 1, characterized in that the inner part of the microcompartment comprises at least one three-dimensional cell aggregate and/or cellular microtissue.

18. An assembly of three-dimensional cellular microcompartments, characterized in that at least one microcompartment is a microcompartment according to claim 1.

19. The cellular microcompartment according to claim 1 or assembly of these cellular microcompartments for use thereof as a medicament.

20. The use of a microcompartment according to claim 1 or assembly of these cellular microcompartments for manufacturing microtissues.

21. A method for preparing a cellular microcompartment according to claim 1, comprising the following steps:
a. mixing cells, optionally previously incubated in a culture medium,
b. encapsulating the mixture from step (a) in a hydrogel intended to form the outer layer of hydrogel
c. cultivating the capsules obtained in step (b) in a culture medium, preferentially in a bioreactor, preferentially for at least 1 day, even more preferentially from 3 to 50 days, and
d. optionally recovering the cellular microcompartments obtained, characterized in that, during step (a) and/or during step (b), a hydrogel solution of plant or synthetic origin is added, the Young's modulus of which is strictly less than the Young's modulus of the hydrogel used to form the outer layer of hydrogel.

22. The method according to claim 21, characterized in that step b) is carried out by simultaneous co-injection of a hydrogel solution intended to form the outer layer (i), of the mixture from step a) optionally comprising the hydrogel solution of plant or synthetic origin whose Young's modulus is strictly less than that of the hydrogel used to form the outer layer (ii), and optionally of an intermediate solution (iii) optionally comprising the hydrogel solution of plant or synthetic origin whose Young's modulus is strictly less than that of the hydrogel used to form the outer layer; said co-injection is performed concentrically via a microfluidic or millifluidic injector forming a jet at the injector outlet made up of the mixture of said solutions, said jet breaking up into drops.

23. The method according to claim 22, characterized in that the final opening diameter of the microfluidic injector is between 50 and 800 μm, preferentially between 80 and 240 μm, and the flow rate of each of the solutions is between 10 and 2000 mL/h, preferentially between 11 and 100 mL/h.

24. The method according to claim 21, characterized in that step b) is carried out by simultaneous co-injection of the hydrogel solution intended to form the outer layer (i), of the mixture from step a) optionally comprising the hydrogel solution of plant or synthetic origin whose Young's modulus is strictly less than that of the hydrogel used to form the outer layer (ii), and optionally of an intermediate solution (iii) optionally comprising the hydrogel solution of plant or synthetic origin whose Young's modulus is strictly less than that of the hydrogel used to form the outer layer; said co-injection is performed concentrically via a microfluidic or millifluidic injector, said injector comprising a tip, said tip being in contact with a calcium solution, forming a jet at the injector outlet consisting of the mixture of said solutions, said jet forming a tube.

25. The method according to claim 24, characterized in that the final opening diameter of the microfluidic injector is between 50 and 1000 μm, preferentially between 80 and 300 μm, and the flow rate of each of the solutions is between 1 and 100 mL/h.

26. The use of a kit for preparing a microcompartment according to one of claim 1, said kit comprising at least one hydrogel solution intended to form the outer layer of the microcompartment (i) and a hydrogel solution of plant or synthetic origin whose Young's modulus is strictly less than that of the hydrogel used to form the outer layer (ii).

27. A peptide sequence comprising
a. a sequence comprising n glycine residues, and
b. at least one YIGSR motif or at least one RGD motif.

28. The peptide sequence according to claim 27, characterized in that it is selected from the group consisting of SEQ ID NO: 1, SEQ ID NO:2, SEQ ID NO:3, SEQ ID NO:4, SEQ ID NO:5 and SEQ ID NO:6.

29. The cellular microcompartment according to claim 1, characterized in that the hydrogel mesh of plant or synthetic origin comprises at least one peptide sequence comprising
a. a sequence comprising n glycine residues, and
b. at least one YIGSR motif or at least one RGD motif.


SEQUENCE LISTING (6 sequences)

SEQ ID NO: 1 - protein, 11 aa, synthetic construct
GGGGGGYIGSR

SEQ ID NO: 2 - protein, 14 aa, synthetic construct
GGGGGGGGGYIGSR

SEQ ID NO: 3 - protein, 17 aa, synthetic construct
GGGGGGGGGGGGYIGSR

SEQ ID NO: 4 - protein, 9 aa, synthetic construct
GGGGGGRGD

SEQ ID NO: 5 - protein, 12 aa, synthetic construct
GGGGGGGGGRGD

SEQ ID NO: 6 - protein, 15 aa, synthetic construct
GGGGGGGGGGGGRGD